(19) United States
(12) Patent Application Publication
(10) Pub. No.: US 2025/0051807 A1
(43) Pub. Date: Feb. 13, 2025

(54) METHOD FOR ENHANCED BIOGAS GENERATION

(71) Applicant: SixRing Inc., Calgary (CA)

(72) Inventors: Corynne O'Farrell, Calgary (CA); Julie Greer, Calgary (CA); Markus Weissenberger, Calgary (CA); Alejandra Enriquez, Calgary (CA)

(21) Appl. No.: 18/437,620

(22) Filed: Feb. 9, 2024

(30) Foreign Application Priority Data
Aug. 11, 2023 (CA) 3209028

(51) Int. Cl. C12P 5/02

(52) U.S. Cl. CPC C12P 5/023; C12P 2203/00

(57) ABSTRACT

In one example, a process to make biogas includes providing a digester which comprises at least one organic material rich in nitrogen and at least one inoculum capable of converting a portion of said at least one organic material into methane under anaerobic conditions; providing a biomass which is depleted of lignin; adding said biomass to said digester; allowing sufficient time for the digester to degrade at least a portion of said biomass and at least a portion of said organic material to yield a biogas composition comprising methane; capturing said biogas composition; and storing said biogas. Also disclosed, are methods and uses associated with the use of a cellulosic component processed to be depleted of lignin which comprises approximately between 10% to 50% of the amount of lignin present in a biomass component prior to a delignification treatment, as additive to organic waste in a bio-digester.

### RELATED APPLICATIONS

This application claims priority to Canadian Patent Application No. 3,209,028, titled “Method for Enhanced Biogas Generation,” filed on Aug. 11, 2023, which is hereby incorporated by reference in its entirety.

### TECHNICAL FIELD

The present invention is directed to the use of a pretreated cellulose in the generation of biogas, more specifically, in the use of a low-lignin content cellulose as an additive or feedstock to organic matter in an anaerobic digester.

### BACKGROUND

Biogases are the product of microbial degradation of various organic materials (both plant-based and animal-based products) in the absence of oxygen, i.e., in an anaerobic environment. This microbial degradation in an oxygen-free environment is called anaerobic digestion. Biogas can refer to gas produced naturally and industrially and generally contains between 45-75 percent methane (CH₄ or natural gas), 25-45 percent carbon dioxide (CO₂), and trace amounts of various other gases.

Natural sources of biogas include environments such as swamps, which generate methane by action of methanogens. A more common source of biogas is related to industrial activities related to waste disposal, principally related to landfills. A second, less widespread, source of biogas from human activities comes from anaerobic digesters and is used to generate methane and recycle organic waste to be used in fertilizing the field. Biogas is generated when organic waste is exposed to microbes, i.e., bacteria, archaea, and fungi, under anaerobic conditions (no oxygen present) to break down the organic compounds. This biodegradation yields gas (biogas), liquids and solids. The latter two, called digestate, can be used as soil amendments in fields for agriculture. The composition and nutrient content of the digestate is greatly impacted by the feedstock that undergoes anaerobic digestion as well as the operation conditions of the digester.

One of the end uses of industrially generated biogas is to burn it to provide a source of heat for buildings, boilers and perhaps even the biodigester. Biogas from landfills and digesters can also be refined to separate the methane (natural gas) from the other non-desirable constituents of biogas such as, but not limited to, carbon dioxide, water vapor, hydrogen sulfide, and others. This refining step yields renewable natural gas (RNG) which can be used as is, can be injected into an existing natural gas grid, or even used for vehicles powered by natural gas.

A significant benefit of biogas recovered from biodigesters, or landfills is the reduction of usage of fossil fuels. Use of biogas generated from biodigesters, or landfills can provide a clean source of power, which also happens to reduce the amount of methane released into the atmosphere. Since natural gas (methane) is a greenhouse gas which is, pound for pound, over 20 times more dangerous to the atmosphere that carbon dioxide over a 20-year period, it is desirable to reduce the release of this gas into the atmosphere. By controlling the release of this gas from its main industrial sources, it is possible to harness the energy it provides all the while providing a green alternative to power generation. Anaerobic digestion provides additional benefits to communities and the environment such as reducing odours, pathogens, improving soil health and reducing the risk of water pollution associated with manure coming from livestock.

The United States currently has over 2,000 biogas systems spread out across the country, but it has the potential to add at least another 10,000 additional plants and thus having a significant positive impact of the environment. In fact, proper harnessing of the potential of biogas in the United States alone would be the equivalent of removing the emissions of millions of cars yearly.

In addition to environmental benefits, the increased implementation of anaerobic digesters using various agricultural and food waste would inevitably reduce the costs associated with waste management if such were simply directed to landfills. Additional advantages of greater implementation of anaerobic digesters in the United States include the building of thousands of biogas systems, which would support hundreds of thousands of construction jobs and the resulting biodigester plants would employ several tens of thousands of people to operate them.

Wastewater treatment plants can have on-site anaerobic digesters to treat sewage sludge recovered during treatment. The solids are separated while the water is released and, more often than not, the methane generated is simply burnt into the atmosphere (i.e., it is flared) without benefiting of the energy this combustion generates. Only roughly two thirds of wastewater treatment plants in the United States that have anaerobic digesters actually use the biogas they generate.

### Biogas Feedstocks

Food waste in landfills is responsible for over 20% of the volume of waste present in U.S. landfills. This food waste is an important source of natural gas as it breaks down. While landfills may capture the resultant biogas, putting organic wastes in landfills does not enable operators to recover the nutrients generated from the source organic material such as fats, oils, and grease collected from the food service industry (added to an anaerobic digester to increase biogas production).

Livestock waste is another important source of methane. An average dairy cow weighing 1000 pounds produces approximately 80 pounds of manure per day, which is a non-negligible source of methane. It was assessed, in 2015, that livestock waste contributed to 10% of all methane emissions in the United States but that only 3% of all manure was actually being recycled in biodigester plants. This is a significant lost opportunity as well as environmentally careless.

Organic waste in landfills produces biogas, which is released into the atmosphere. Methane generated from landfills are ranked as the third largest source of such gas (by volume) related to human activities in the United States. Microorganisms present in landfills are similar to those found in bio-digesters, in that they are capable of breaking down organic materials and generating biogas. As methane is a potent greenhouse gas, it is desirable to capture these emissions to utilize them as a potential source of energy.

Crop residues are a source of organic material which can be processed in an anaerobic digester. These residues are meant to include, but not be limited to, stalks, straw, and plant trimmings. There are sufficient crop residues to leave a portion on the field in order to reduce the amount of soil erosion and harvest the remainder for biogas production. It is estimated that there is over 100 million tons of crop residues available which could be used in biogas systems. One drawback of crop residues is that they contain lignin which is very poorly digested in biodigesters. Crop residues are typically mixed with a variety of other organic materials to generate biogas.

### Composition of Biogas

The composition of biogas is dependent on the feedstock, as well as the conditions of the biodigester such as temperature, pH, organic loading rate, etc. Typical biogas composition is made up of 45-75% methane, 25-45% carbon dioxide and 5% or less of various other gases.

Commercial scale anaerobic biodigesters face a number of different issues, with the most common being feedstock variability, low process efficiency, and lower product quality. These issues can be really significant for the asset owner or operator as it could not only translate to a non-economically favorable digester but could have considerable safety hazards to operators and the community (i.e., generation of offensive odours and/or generation of increased amounts of hydrogen sulfide, H₂S). Currently, there are a wide range of techniques available to anaerobic digester operators and owners to overcome these challenges. Pretreatment of highly recalcitrant feedstocks has been utilized to improve the hydrolysis rate of certain types of biomasses that are more difficult to degrade by the microbial community present in the anaerobic digester. These pretreatments can be categorized as mechanical pretreatments (such as grinding the biomass, chipping, cavitation, mechanical refining, deflaking, dispersing and use of a Hollander beater), thermal pretreatments (hydrothermal, microwave, extrusion, torrefaction, steam explosion, and wet oxidation), chemical pretreatments (which covers acidic, basic, redox reactions and ionic liquids), and combinations thereof. However, in most cases the high capital cost, high consumption of energy and chemicals, low delignification efficiencies, and sophisticated operating conditions are the major factor hindering their full-scale application.

Lignocellulosic biomass is a widely available resource which can be used in biogas production. When lignin is present in the unprocessed biomass, or in a pulp after incomplete delignification, and it is used as part of the feedstock for a biodigester, it results in a reduction of the rate of hydrolysis of said feedstock by the microbial community. This is due to lignin being highly recalcitrant to biodegradation, especially under anaerobic conditions. It has been observed that there is an inverse relationship between the amount of lignin in plant biomass and the corresponding biomethane potential, in which lignin limits the bioavailability of the more readily degradable cellulose since, as they are tightly linked together. Lignin, and its corresponding dissolved lignin components, will accumulate as the microbial community preferentially targets the more readily biodegradable compounds, which will eventually limit certain microbial activities and hinder the efficiency of other processes required for adequate anaerobic digestion. As such, it is preferable to minimize the amount of lignin remaining in the feedstock when the latter is used in anaerobic digestion for the generation of biogas in order to maximize the methane yield.

In addition to that, in jurisdictions where waste lignocellulosic biomass is limited, operators and asset owners have had to resort to the use of very recalcitrant, hard-to-degrade feedstocks (i.e., higher lignin content, high cellulose crystallinity, etc.). These have proven to be more difficult to incorporate in anaerobic digesters, resulting in inefficient biogas generation. Lignin has a complex aromatic and highly branched structure and is therefore not favoured by microorganisms. This complex structure blocks access to the cellulose part of the biomass that would normally be readily degradable. Lignin is not an ideal source of carbon for biogas generation due to the numerous degradation reactions that must occur making it a time-consuming process that few organisms are capable of. Also, as lignin is biodegraded, it produces oxidants and phenolic compounds, which will act as anti-microbials, hindering other members of the microbial community present in the anaerobic digesters.

In light of the above, it is clear that biogas production needs to increase in the coming years in order to reduce countries dependence on oil and fossil fuel-based products, especially given the fact that such biogas facilities can be readily implemented. However, in order to optimize biogas production from such biogas facilities it is desirable to be able to provide a more consistent biogas output which is rich in methane. The composition of the biogas generated at such facilities may vary during the year depending on the available feedstock which is used. In many areas, farmers having a biogas facility on their farms or close by are able to provide some of the feedstock. However, some feedstock is not necessarily available year-round or scarce and thus, changing the composition of the feedstock over the year has a direct impact on the generation of biogas and the composition thereof.

Because of these factors, it is desirable to provide an alternative additive (or feedstock) which will supplement at least a portion of the organic material being used in the production of biogas or at least help in increasing or stabilizing methane production.

### SUMMARY

According to a first aspect of the present invention, there is provided a method to increase methane production from an anaerobic digester, wherein said method comprises the addition of a component (either as additive or feedstock) which is depleted of lignin.

According to a preferred embodiment of the present invention, the component is a cellulosic material which has been processed to be depleted of lignin.

The terms “depleted of lignin”, and “lignin-depleted” as used herein refers to a cellulosic component resulting from the delignification or pretreatment of a lignocellulosic biomass wherein the lignin content post-treatment ranges between 2 to 15%. Preferably, between 2 to 10%.

Many different types of organic materials can be fed to anaerobic digesters. In general, and for the purposes of this invention, “feed” is referred to as the overall material added to the anaerobic digester or similar vessel at an established frequency (i.e., daily). The “feed” is typically comprised of two components. A first component, which is an inoculum comprising a microbial community capable of breaking down organic matter and producing biogas. This can be in the form of manure (bovine, porcine, or equine) or in the form of sewage sludge. A second component comprises of organic materials, referred to as “feedstock” herein. Examples of said organic materials are food by-products, agricultural and slaughterhouse waste products, fats, oils, greases, industrial organic residues, waste lignocellulosic biomass, etc.

According to a preferred embodiment of the present invention, the biodigester is loaded with the following:
a first component comprising at least one inoculum capable of converting a portion of said at least one organic material into methane under anaerobic conditions;
a second component comprising at least one organic material rich in nitrogen;
a third component comprising a treated biomass component whose initial lignin content has been depleted by an amount ranging from 50% up to 90% by a prior treatment step (such as a delignification reaction), wherein said treated biomass component is present in an amount that range between 0.1 to 5% w/w of the weight of said feedstock.

According to another aspect of the present invention, there is provided a process to make biogas, said process comprising the steps of:
providing a digester adapted to receive a feed and capture a biogas composition resulting from anaerobic digestion;
providing said feed comprising:
   a first component comprising at least one inoculum comprising a microbial community;
   a feedstock component comprising:
      a second component comprising at least one organic material rich in nitrogen; and
      a third component comprising a treated biomass component whose initial lignin content has been depleted by an amount ranging from 50% up to 90% by a prior treatment step (such as a delignification reaction), wherein said treated biomass component is present in an amount that range between 0.1 to 5% w/w of the weight of said feedstock;
         wherein said at least one inoculum is capable of converting a portion of said at least one organic material into methane under anaerobic conditions;
adding said feed to said digester;
allowing sufficient time for the feedstock to be degraded wherein at least a portion of said third component and at least a portion of said second component is converted to a biogas composition comprising methane;
optionally, continuously adding said feed comprising said biomass which is depleted of lignin in an amount that ranges between 0.1 to 5% w/w of the total feedstock comprising at least one other organic material.
capturing said biogas composition; and
optionally, storing at least a portion of said biogas.

According to another aspect of the present invention, there is provided a method to increase and stabilize the volume of methane produced from an anaerobic digester by using a treated lignocellulosic biomass component as additive or partial replacement in a biodigester adapted to generate methane from organic waste (organic feed) used for biogas production. Preferably, said treated lignocellulosic biomass component comprises 10% or more of the amount lignin present prior to a lignocellulosic biomass undergoing a delignification treatment. More preferably, said treated lignocellulosic biomass component comprises 15% to 25% of the amount lignin prior to a lignocellulosic biomass undergoing a delignification treatment.

According to a preferred embodiment of the present invention, said treated lignocellulosic biomass component is present in an amount ranging from 0.1% w/w to 5% w/w of the total organic feedstock in the bio-digester. Preferably, said treated lignocellulosic biomass component is present in an amount ranging from 0.5% w/w to 2.5% w/w of the total organic feedstock in the bio-digester. More preferably, said treated lignocellulosic biomass component is present in an amount ranging from 0.5% w/w to 1% w/w of the total organic feedstock in the bio-digester.

According to another aspect of the present invention, there is provided a process to make biogas, said process comprising the steps of:
providing a digester adapted to receive a feed and capture a biogas composition resulting from anaerobic digestion;
providing said feed comprising:
   at least one inoculum capable of converting a portion of said at least one organic material into methane under anaerobic conditions;
   at least one organic material rich in nitrogen also referred to as feedstock;
   a treated biomass component whose initial lignin content has been depleted by an amount ranging from 50% up to 90% by a prior treatment step such as a delignification reaction, wherein said treated biomass component is present in an amount that range between 0.1 to 5% w/w of the weight of said feedstock;
adding said feed to said digester;
allowing sufficient time for the digester to degrade at least a portion of said treated biomass component and at least a portion of said organic material rich in nitrogen to yield a biogas composition comprising methane;
optionally, continuously adding said feed comprising said biomass which is depleted of lignin in an amount that ranges between 0.1 to 5% w/w of the total feedstock comprising at least one other organic material rich in nitrogen;
capturing said biogas composition; and
optionally, storing at least a portion of said biogas.

According to yet another aspect of the present invention, there is provided a process to make biogas, said process comprising the steps of:
providing a digester adapted to receive a feed and capture a biogas composition resulting from anaerobic digestion;
adding to said digester at least one organic material rich in nitrogen and at least one inoculum capable of converting a portion of said at least one organic material into methane under anaerobic conditions;
providing a cellulose which has been processed to be depleted of lignin which comprises approximately between 10% to 50% of the amount of lignin present in a biomass component prior to a delignification treatment, said cellulose is present in an amount that ranges between 0.1 to 5% w/w of the total feedstock, comprising at least one other organic material rich in nitrogen;
adding said cellulose to said digester;
allowing sufficient time for the digester to degrade at least a portion of said biomass and at least a portion of said organic material to yield a biogas composition comprising methane;
optionally, continuously adding said cellulose in an amount that ranges between 0.1 to 5% w/w of the total feedstock comprising at least one other organic material rich in nitrogen;
capturing said biogas composition; and
optionally, storing said biogas.

According to another aspect of the present invention, there is provided a process to make biogas, said process comprising the steps of:
providing a digester adapted to receive a feed and capture a biogas composition resulting from anaerobic digestion;
providing said feed comprising:
   at least one inoculum capable of converting a portion of said at least one organic material into methane under anaerobic conditions;
   at least one organic material rich in nitrogen also referred to as feedstock;
   a biomass whose lignin content has been depleted by an amount ranging from 50% up to 90% by a prior treatment step such as a delignification reaction, wherein said biomass is present in an amount that range between 5 to 100% w/w of the weight of feedstock;
adding said feed to said digester;
allowing sufficient time for the feed to be degraded wherein at least a portion of said biomass and at least a portion of said organic material rich in nitrogen to yield a biogas composition comprising methane;
optionally, continuously adding said feed comprising said biomass which is depleted of lignin in an amount that ranges between 5 to 100% w/w of the total feedstock;
capturing said biogas composition; and
optionally, storing at least a portion of said biogas.

According to yet another aspect of the present invention, there is provided a process to increase the amount of methane produced from an anaerobic digester, said method comprising the steps of:
providing a digester adapted to receive a feed and capture a biogas composition resulting from anaerobic digestion;
adding to said digester at least one organic material rich in nitrogen and at least one inoculum capable of converting a portion of said at least one organic material into methane under anaerobic conditions;
providing a cellulose which has been processed to be depleted of lignin which comprises approximately between 10% to 50% of the amount of lignin present in a biomass component prior to a delignification treatment, said cellulose is present in an amount that ranges between 5 to 100% w/w of the total feedstock;
adding said cellulose to said digester;
allowing sufficient time for the digester to degrade at least a portion of said biomass and at least a portion of said organic material rich in nitrogen to yield a biogas composition comprising methane;
optionally, continuously adding said cellulose in an amount that ranges between 5 to 100% w/w of the total feedstock;
capturing said biogas composition; and
optionally, storing said biogas.

According to yet another aspect of the present invention, there is provided a process to increase the amount of methane produced from an anaerobic digester, said method comprising the steps of:
providing a digester adapted to receive a feed and capture a biogas composition resulting from anaerobic digestion;
adding to said digester an organic matter which comprises at least one organic material nitrogen-rich component and at least one inoculum capable of converting a portion of said at least one organic material into methane under anaerobic conditions;
providing a cellulose which has been processed to be depleted of lignin which comprises approximately between 10% to 50% of the amount of lignin present in a biomass component prior to a delignification treatment, said cellulose is present in an amount that ranges between 5 to 100% w/w of the total feed;
adding said cellulose to said digester;
allowing sufficient time for the digester to degrade at least a portion of said biomass and at least a portion of said organic material rich in nitrogen to yield a biogas composition comprising methane;
optionally, continuously adding said cellulose is present in an amount that ranges between 5 up to, but not including, 100% w/w of the total feed;
capturing said biogas composition; and
optionally, storing said biogas.

According to yet another aspect of the present invention, there is provided a use of a cellulosic component which has been processed to be depleted of lignin which comprises approximately between 10% to 50% of the amount of lignin present in a biomass component prior to a delignification treatment, as additive to organic material or feedstock intended for biogas production to increase the volume of methane produced from a biogas digester, wherein said cellulosic component is added in an amount not exceeding 5% w/w of the total feed in the biogas digester. Preferably, said cellulosic component stabilizes the volume of methane produced from a biogas digester.

According to yet another aspect of the present invention, there is provided a use of a cellulosic component which has been processed to be depleted of lignin which comprises approximately between 10% to 50% of the amount of lignin present in a biomass component prior to a delignification treatment as an additive to organic waste intended for biogas production to decrease the volume of carbon dioxide produced from a biogas digester, wherein said cellulosic component is added in an amount not exceeding 5 w/w % of the total feed in the biogas digester.

According to yet another aspect of the present invention, there is provided a use of a cellulosic component which has been processed to be depleted of lignin which comprises approximately between 10% to 50% of the amount of lignin present in a biomass component prior to a delignification treatment, as a feedstock intended for biogas production to increase the volume of methane produced from a biogas digester, wherein said cellulosic component is added in an amount ranging from 5 to 100 w/w % of the total feed in the biogas digester. Preferably, said cellulosic component stabilizes the volume of methane produced from a biogas digester.

According to yet another aspect of the present invention, there is provided a use of a cellulosic component which has been processed to be depleted of lignin which comprises approximately between 10% to 50% of the amount of lignin present in a biomass component prior to a delignification treatment as a feedstock intended for biogas production to decrease the volume of carbon dioxide produced from a biogas digester, wherein said cellulosic component is added in an amount ranging from 5 to 100 w/w % of the total feed in the biogas digester.

According to yet another aspect of the present invention, there is provided a process to make biogas, said process comprising the steps of:
providing a digester adapted to receive a feed and capture a biogas composition resulting from anaerobic digestion;
providing said feed comprising:
   at least one inoculum capable of converting a portion of an organic matter which comprises at least one organic material nitrogen-rich component into methane under anaerobic conditions;
   a biomass whose lignin content has been depleted by an amount ranging from 50% up to 90% by a prior treatment step such as a delignification reaction, wherein said biomass is present in an amount that range between 5 to 100% w/w of the weight of the feedstock
   optionally, at least one organic material rich in nitrogen to make up the rest of the feedstock;
adding said feed to said digester;
allowing sufficient time for the feedstock to be degraded wherein at least a portion of said biomass and at least a portion of said organic material rich in nitrogen to yield a biogas composition comprising methane;
optionally, continuously adding said feed comprising said biomass which is depleted of lignin in an amount that ranges between 5 to 100% w/w of the total feedstock optionally comprising at least one other organic material rich in nitrogen;
capturing said biogas composition; and
optionally, storing at least a portion of said biogas.

It will be understood by the person skilled in the art that biogas is generated after organic materials (plant and animal products) are broken down when exposed to fungi, bacteria and archaea, in an anoxic environment (i.e., under anaerobic conditions). This is also referred to as anaerobic digestion. Anaerobic digestion of organic material yields biogas and residual solids and liquids which is called the digestate. The digestate is rich in nutrients that were present in the original organic material but is now more readily available for plants and soil. The composition and nutrient content of the digestate is determined by the type of feedstock used in the decomposition of the organic matter added to the digester.

According to a preferred embodiment of the present invention, there is provided a method to generate biogas which employs a lignin-depleted cellulosic component obtained from the exposure of a lignocellulosic feedstock to a delignification or pulping process to remove most of the hemicellulose and/or lignin.

The biogas comprises several gases including, but not limited to, methane, carbon dioxide, hydrogen sulfide, and volatile fatty acids. In most cases, the residual solids and liquids (“digestate”) can be used as fertilizer for soils.

### BRIEF DESCRIPTION OF THE FIGURES

Features and advantages of embodiments of the present application will become apparent from the following detailed description and the appended figures, in which:

FIG. 1 is a graphical depiction of the cumulative methane produced in bottles containing a low lignin cellulose derived from hardwood compared with cumulative methane produced in bottles containing raw hardwood biomass; and

FIG. 2 is a graphical depiction of the cumulative methane production of the samples as per Experiment #3.

### DETAILED DESCRIPTION

One of the technical barriers to greater adoption of biogas as an alternative fuel source is related to infrastructural challenges. One of such challenges is the variance in the availability of the feedstock used in biodigesters. It is known that various organic materials may be added to a biodigester and produce biogas, however, what is not as well understood or appreciated is that there are several gases which are formed during such anaerobic digestion. The varying composition of the biogas is correlated to the feedstock (organic material) added for anaerobic digestion. The availability of feedstock being of organic nature naturally varies throughout the year as based on the growing seasons. Moreover, the winter season is generally associated with a reduced biogas production.

According to a preferred embodiment of the present invention, there is provided a method to increase the methane production from a biogas digester.

It has been surprisingly found that when using a lignin-depleted cellulosic component in an anaerobic digester, the methane gas volume produced increases as compared to a non-delignified biomass substrate.

According to a preferred embodiment of the present invention, there is provided a method to increase and stabilize the volume of methane produced from a biogas digester by using a lignin-depleted cellulosic component for biogas production. It has been surprisingly found that adding a lignin-depleted cellulosic component to an anaerobic digester in the presence of other organic materials can increase the methane gas volume produced.

According to a preferred embodiment of the present invention, the composition of gases comprises at least 60% methane to be used as an efficient source of energy.

Currently, biomass fed to anaerobic digesters is not delignified and the presence of lignin causes inhibition of the biodegradation of said biomass by microorganisms present in the biodigester and consequently hinder optimal methane production from such units. Preferably, by using cellulose obtained from a delignification process as an additive (or feedstock) to anaerobic digesters, the cellulose is in a more readily accessible form for the microorganisms in the biodigester.

According to a preferred embodiment of the present invention, the cellulosic component used in anaerobic digestion is a cellulose which has been processed to have between 1 and 10% w/w of lignin.

Preferably, the addition of a lignin-depleted cellulosic component allows for an increase in the generation of methane in a biodigester when all or part of the feedstock is replaced with said lignin-depleted cellulose. Preferably, said lignin-depleted cellulosic component is cellulose and is present in an amount ranging from 5% w/w to 100% w/w of the total feedstock added daily in the biodigester, considering the feedstock is one of two components added to anaerobic digestion. Preferably, the cellulose present in the lignin-depleted cellulosic component is hydrated, in some cases the water may be up to 90% of the weight of the cellulose. According to a preferred embodiment, the cellulose is present in an amount ranging from 10 to 50% w/w of the total organic feedstock in the biodigester. According to a more preferred embodiment of the present invention, the cellulose is present in an amount ranging from 12.5 to 40% w/w of the total organic feedstock in the biodigester. According to yet another preferred embodiment, the cellulose is present in an amount ranging from 15% w/w to 30% of the total organic feedstock in the biodigester. Preferably, the amount of lignin-depleted cellulosic component may be adjusted based on the composition of the organic content present in the digester. It is also desirable that given the possible fluctuations between various biodigesters (due to their different organic content and microbial communities), a pre-determination be done to assess the optimal concentration of the lignin-depleted cellulosic component to be incorporated with the other organic content inside the digester so as not to incorporate an amount which is not optimal as the component may be more costly than the other organic content inside the bio-digester.

In another preferred embodiment of the present invention, the addition of a lignin-depleted cellulosic component is employed as an additive to anaerobic digestion. Said additive increases methane volume production when used as a small portion of the feedstock. Said additive is present in an amount ranging from 0.1 to 5.0% w/w of the total organic feedstock in the biodigester. According to yet another preferred embodiment, the cellulose is present in an amount of approximately 1% w/w of the total organic feedstock in the biodigester. Preferably, the amount of biomass additive may be adjusted based on the composition of the organic content present in the digester.

It is generally accepted that biogas is formed mainly by the degradation of organic materials such as: carbohydrates, proteins, and lipids. The lignin fraction present in various materials added to digesters is known to be difficult to degrade by the microorganisms present in the digesters. While anaerobic digestion of lignin has been observed in certain environments, it requires a variety of microorganisms whose main pathway involves the enzymatic depolymerization of lignin. Even when these organisms are present, the process tends to be slow. Because of the complexity of the microbial community present in those systems, they are very difficult to reproduce on an industrial scale, making the applicability of the anaerobic digestion of lignin a complicated and difficult topic.

By adding a cellulose-rich material whose lignin content has been reduced, it has been made possible to increase the generation of methane in a biodigester.

According to a preferred embodiment of the present invention, it is desirable, to balance out the nitrogen-rich material with cellulose-rich material in order to achieve a carbon to nitrogen ratio of the feed ranging preferably between 20:1 and 30:1 to obtain optimal methane generation. High nitrogen content organic materials also referred to as organic material rich in nitrogen include manure, crops and crop waste, grass trimmings, animal by-products, ground coffee, etc. Low nitrogen content organic material include bark, wood chips, sawdust, straw, etc.

When loading the anaerobic digester, the operator must consider and be conscious of the feed which is inputted therein. There must be some consideration placed on the type of material used and ultimately its impact on the carbon to nitrogen ratio as this will directly impact the performance of the biodigester and its methane production.

Experiment #1-Determination of Theoretical Methane Potential (TMP) of Raw Hardwood and a Lignin Depleted Cellulose Derived from Hardwood.

The purpose of this experiment was to determine the TMP of raw hardwood biomass and compare it with the TMP of a lignin-depleted cellulose derived from hardwood, whose lignin had been partially removed using the Kraft process. Raw hardwood biomass was milled to 1 mm in size and the lignin-depleted cellulose was blended in a blender. These substrates were then suspended in water and analyzed for Chemical Oxygen Demand (COD) determination. COD is a measure of the amount of oxygen that can be consumed by reactions during the decomposition of organic matter suspended in water. This value describes how much energy is available in the organic matter and can be used to calculate the theoretical amount of methane that could be produced from the complete decomposition of that organic matter. The same samples were characterized by testing their total solids (TS) as well as volatile solids (VS). This was performed as per US EPA method 1684. The % of volatile solids is a measure of the amount of material lost after a sample is ignited. The results of these tests are reported in Table 1.

TABLE 1
Theoretical methane potential for raw hardwood biomass
and lignin-depleted cellulose derived from hardwood.
Calculated based on COD and VS for Experiment #1

| | Chemical | | Theoretical |
|---|---|---|---|
| | Oxygen | | Methane |
| | Demand | Volatile | Potential |
| Substrate | (mg/L) | Solids (%) | (mL/g VS) |
| Lignin-depleted | 11700 | 31.70 | 129 |
| cellulose | | | |
| Hardwood biomass | 8600 | 94.59 | 67 |

The results of this experiment show an almost 2-fold increase in TMP in the lignin-depleted hardwood cellulose substrate when compared to the raw hardwood biomass. This suggests that the amount of methane that can be generated from a lignin-depleted cellulose would be almost twice that of a raw hardwood biomass when degraded in an anaerobic digestion system, when the same amounts were added.

Experiment #2-Determination of the Biomethane Potential (BMP) of Raw Hardwood and a Lignin-Depleted Cellulose Derived from Hardwood.

The purpose of this experiment was to determine and compare the BMP of raw hardwood biomass and a lignin-depleted cellulose sample derived from hardwood, whose lignin had been in great part removed via the Kraft process. This experiment was designed to corroborate the theoretical results obtained from Experiment #1.For the experiment preparation, the Total Solids (TS) and Volatile Solids (VS) were measured for all substrates and the inoculum (digestate). Inoculum was prepared by incubating it under anaerobic conditions for six days prior to setting up the serum bottles. Substrates were added to serum bottles containing the prepared inoculum in an inoculum-to-substrate ratio of 2.5 based of the volatile solids of the materials. Corresponding substrate-free blank bottles were prepared by adding water in place of the substrate. Bottles were incubated at 40° C. for the duration of the experiment. Biogas and methane production of test samples were measured three times a week or as needed.

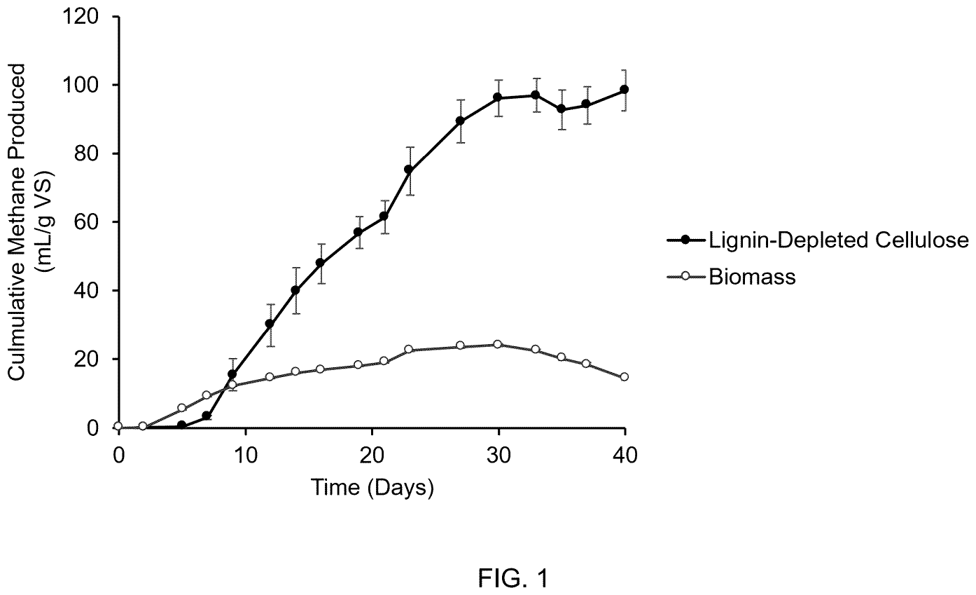

For analysis, the methane generated from the substrate-free blanks was subtracted from the methane generated from the experimental substrates. This was done to ensure that the result is a reflection of the amount of methane produced from the addition of the substrate alone. The results for this experiment are shown in Table 2 and FIG. 1.

TABLE 2
Maximum Biochemical Methane Potential (from
FIG. 1) as well as corresponding lignin content
of the substrates tested in Experiment #2

| Substrate | BMP (mL CH₄/g VS) | Lignin content (% OD) |
|---|---|---|
| Lignin-depleted | 98.36 | 2.6 |
| cellulose | | |
| Hardwood biomass | 24.09 | 19.8 |

This experiment has substantiated the advantage of using any type of mechanical or chemical pretreatment to lower the lignin content in materials as a pretreatment of a traditionally recalcitrant biomass such as hardwood for use in an anerobic digester system. After a period of 40 days, there was a distinct plateau in the lignin-depleted cellulose bottles at around 100 mL of methane per g of VS while the hardwood biomass plateaued at approximately 20 mL of methane per gram VS by day 21. The amount of methane produced per gram of VS from the lignin-depleted cellulose was almost 5 times more than the amount of methane produced per gram VS of the raw hardwood biomass. The hardwood biomass BMP result was much lower than the TMP results from Experiment #1 while the lignin-depleted cellulose BMP result was similar to the TMP results from Experiment #1.This suggests that the lignin-depleted cellulose was degraded and transformed to methane to its full potential, while the hardwood biomass was not able to fully degrade and convert to methane. This can likely be attributed to an accumulation of inhibitory compounds present in the non-delignified biomass, as TMP cannot account for this.

### Experiment #3-Determination of the Effect of the Addition of a Lignin-Depleted Substrate as a Component of the Feedstock.

The purpose of this experiment was to determine whether the addition of a substrate, whose lignin had been in great part removed via a chemical and/or mechanical process, produced more methane than without the addition of this component. For the experimental design, the control samples contained a feed composed of manure and agricultural feedstock. Said agricultural feedstock was made up of starch products, animal derived protein, and crop waste and thus, high in nitrogen content, with a carbon to nitrogen ratio (C:N)<20. The experimental samples contained lignin-depleted substrates in the form of Kraft cellulose, newspaper or cardboard, replacing a portion of the nitrogen-rich agricultural feedstock. This allows for the determination of the impact of the addition of lignin-depleted cellulosic material alongside a nitrogen-rich component in comparison with the addition of just the nitrogen-rich component in the feed. Table 3 shows the Kappa numbers of the lignin-depleted substrates used for Experiment #3.

TABLE 3
Kappa numbers for all the lignin depleted
substrates used for Experiment #2

| | Kraft Cellulose | Cardboard | Newspaper |
|---|---|---|---|
| Kappa number | 17.5 | 34.4 | 87.1 |

Serum bottles were used to conduct anaerobic digestion experiments. The serum bottles were set up by adding manure, digestate from a commercial scale anaerobic digester, and feedstock comprised of agricultural waste and/or a lignin-depleted cellulosic material. Each serum bottle batch was set to run under mesophilic conditions. The lignin-depleted cellulosic material was added to some of the bottles, partially replacing a portion of the agricultural feedstock.

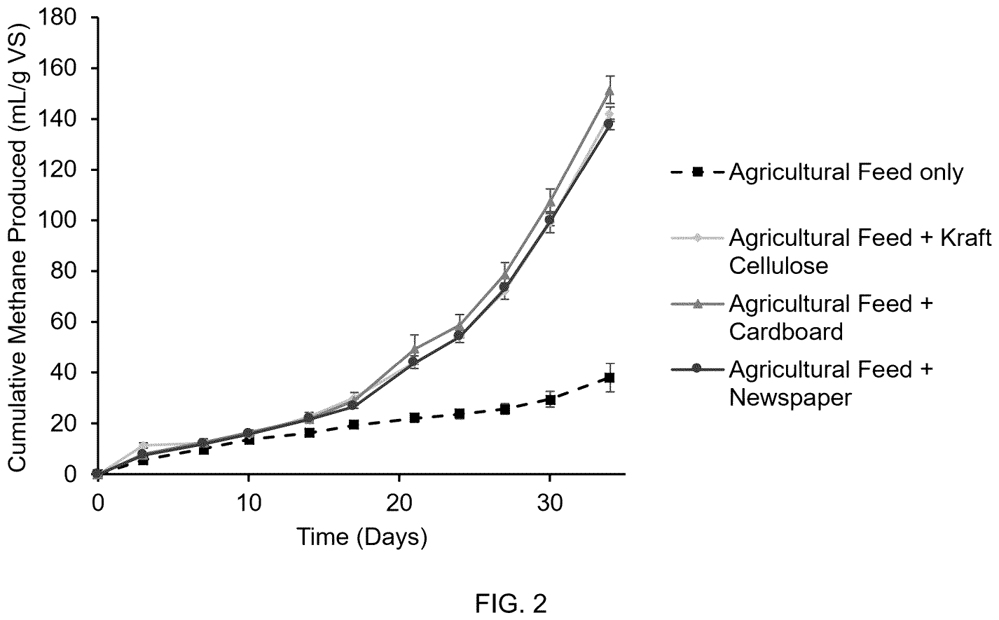

The lignin-depleted cellulosic material was added in an amount so the substrate loading was equivalent to 18% w/w of the feed, while the rest of the organic matter (feedstock) comprised at least one organic material nitrogen-rich component, with a carbon to nitrogen ratio (C:N)<20. The substrate was added to serum bottles in a 20% w/w solids slurry. Biogas produced was measured every 2-3 days and methane concentration was measured twice a week using a GC-FID. The results are reported in Table 4 below and in FIG. 2.

TABLE 4
Cumulative methane generated and standard error
data for Experiment #3 for all feedstocks

| | | Agricultural | Agricultural | Agricultural |
|---|---|---|---|---|
| | Agricultural | Feed + | Feed + | Feed + |
| Day | Feed only | Kraft Cellulose | Cardboard | Newspaper |
| 0 | 0.00 ± 0.00 | 0.00 ± 0.00 | 0.00 ± 0.00 | 0.00 ± 0.00 |
| 3 | 5.55 ± 0.56 | 11.35 ± 1.05 | 7.81 ± 0.50 | 7.44 ± 0.18 |
| 7 | 9.91 ± 0.68 | 12.25 ± 1.60 | 12.59 ± 0.29 | 11.80 ± 0.15 |
| 10 | 13.56 ± 0.94 | 15.56 ± 1.91 | 16.35 ± 0.26 | 15.69 ± 0.48 |
| 14 | 16.28 ± 1.09 | 22.52 ± 1.84 | 21.66 ± 0.14 | 21.50 ± 0.37 |
| 17 | 19.26 ± 1.27 | 29.86 ± 2.16 | 28.83 ± 0.81 | 26.45 ± 0.48 |
| 21 | 22.00 ± 1.72 | 44.00 ± 2.52 | 49.26 ± 5.47 | 43.75 ± 0.58 |
| 24 | 23.62 ± 1.84 | 54.17 ± 2.44 | 58.73 ± 4.02 | 53.99 ± 0.40 |
| 27 | 25.68 ± 2.11 | 72.04 ± 3.31 | 78.68 ± 4.65 | 72.90 ± 0.68 |
| 30 | 29.43 ± 3.12 | 99.11 ± 4.17 | 107.42 ± 4.85 | 99.52 ± 1.70 |
| 34 | 38.03 ± 5.63 | 141.78 ± 2.87 | 151.40 ± 5.39 | 137.73 ± 2.12 |

This experiment has substantiated the advantage of using a lignin-depleted cellulosic material as a component of the feedstock to an anaerobic digester system. In fact, the amount of methane generated through the addition of 18% of the total weight of the feed (comprising manure and feedstock) indicates the propensity of the microorganisms present in the system to generate increased amount of methane when provided with a cellulose-rich and lignin-depleted material.

After a period of 34 days, there was over 3.5 times more methane generated in all samples containing the lignin-depleted cellulosic substrate than in the controls with non-pretreated hardwood. It is also worthy of note that the increase in methane generation was disproportional with respect to the amount of cellulose added to the samples.

While the foregoing invention has been described in some detail for purposes of clarity and understanding, it will be appreciated by those skilled in the relevant arts, once they have been made familiar with this disclosure that various changes in form and detail can be made without departing from the true scope of the invention in the appended claims.

1. A process to make biogas, said process comprising:
providing a digester adapted to receive a feed and capture a biogas composition resulting from anaerobic digestion;
providing said feed comprising:
a first component comprising at least one inoculum comprising a microbial community;
a feedstock component comprising:
a second component comprising at least one organic material preferably rich in nitrogen; and
a third component comprising a treated biomass component whose initial lignin content has been depleted by an amount ranging from 50% up to 90% by a prior treatment step, wherein said treated biomass component is present in an amount that range between 0.1 to 5% w/w of the weight of said feedstock,
wherein said at least one inoculum is capable of converting a portion of said at least one organic material into methane under anaerobic conditions;
adding said feed to said digester;
allowing sufficient time for the feedstock to be degraded wherein at least a portion of said third component and at least a portion of said second component is converted to a biogas composition comprising methane;
optionally, continuously adding said feed comprising said biomass which is depleted of lignin in an amount that ranges between 0.1 to 5% w/w of the total feedstock comprising at least one other organic material;
capturing said biogas composition; and
optionally, storing at least a portion of said biogas.

2. The process of claim 1, further comprising continuously adding said feed comprising said biomass which is depleted of lignin in an amount that ranges between 0.1 to 5% w/w of the total feedstock comprising at least one other organic material.

3. The process of claim 1, further comprising storing at least a portion of said biogas.

4. A process to increase the amount of methane produced from a bio-digester, said method comprising:
providing a digester adapted to receive a feed and capture a biogas composition resulting from anaerobic digestion;
adding to said digester at least one organic material rich in nitrogen and at least one inoculum capable of converting a portion of said at least one organic material into methane under anaerobic conditions;
providing a cellulose which has been processed to be depleted of lignin which comprises approximately between 10% to 50% of the amount of lignin present in a biomass component prior to a delignification treatment, said cellulose is present in an amount that ranges between 0.1 to 5% w/w of the total feed, comprising at least one other organic material;
adding said cellulose to said digester;
allowing sufficient time for the digester to degrade at least a portion of said biomass and at least a portion of said organic material rich in nitrogen to yield a biogas composition comprising methane;
optionally, continuously adding said cellulose in an amount that ranges between 0.1 to 5% w/w of the total feed comprising at least one other organic material;
capturing said biogas composition; and
optionally, storing said biogas.

5. The process of claim 4, further comprising continuously adding said cellulose in an amount that ranges between 0.1 to 5% w/w of the total feed comprising at least one other organic material.

6. The process of claim 4, further comprising storing said biogas.

7. A use of a cellulosic component which has been processed to be depleted of lignin which comprises approximately between 10% to 50% of the amount of lignin present in a biomass component prior to a delignification treatment, as additive to agricultural/organic waste intended for biogas production to increase the volume of methane produced from a biogas digester, wherein said cellulosic component is added in an amount not exceeding 5% w/w of the total daily feedstock added to the anaerobic digester.

8. The use according to claim 7, wherein said cellulosic component stabilizes the volume of methane produced from a biogas digester.

9. The use according to claim 7, wherein said cellulosic component decreases the volume of carbon dioxide produced from a biogas digester.

10. A method to increase and stabilize the volume of methane produced from an anaerobic digester by using a treated lignocellulosic biomass component as partial replacement of the feed in an anaerobic digester adapted to generate methane from organic waste or feed used for biogas production, wherein said treated lignocellulosic biomass component comprises from 10% to 50% of the amount lignin present prior to a lignocellulosic biomass undergoing a delignification treatment.
